(19) United States
(12) Patent Application Publication
(10) Pub. No.: US 2017/0105881 A1
(43) Pub. Date: Apr. 20, 2017

(54) ABSORBENT ARTICLE HAVING AN OUTER BLOUSE LAYER

(71) Applicant: The Procter & Gamble Company, Cincinnati, OH (US)

(72) Inventor: Sarah Marie Wade, Springfield Township, OH (US)

(21) Appl. No.: 15/298,544

(22) Filed: Oct. 20, 2016

Related U.S. Application Data
(60) Provisional application No. 62/243,959, filed on Oct. 20, 2015.

(51) Int. Cl. A61F 13/15; A61F 13/49

(52) U.S. Cl. CPC A61F 13/15203; A61F 13/49011; A61F 2013/15552; A61F 2013/49025; A61F 2013/49092; A61F 2013/15243

(57) ABSTRACT

An absorbent article comprising a liquid permeable topsheet, a liquid impermeable backsheet, an absorbent core structure disposed between the topsheet and backsheet, and an outer blouse layer formed of a nonwoven web, the blouse layer having a blouse layer surface area, the blouse layer being attached to the article at attachment zones having an attachment surface area, the attachment surface area comprising less than 50 percent of the blouse layer surface area.

### BACKGROUND OF THE INVENTION

Current technologies utilized in the materials and construction of disposable absorbent articles such as disposable diapers and disposable absorbent pants have resulted in structures that are thinner, less bulky and more closely and snugly fitting than products of prior years. These features are reflected in the appearance of the articles when worn by, e.g., babies and young children.

In addition to providing a slim and snugly-fitting appearance, these features have resulted in relatively fewer gaps and less air space between the wearer's body and the inner wearer-facing layers of the article. As a result, there is less air movement over the wearer's skin in areas covered by the garment. This may create a greater possibility of diaper rash resulting from or exacerbated by overhydration of the wearer's skin.

Consequently, there is room for improvement in appearance and breathability in such articles.

### BRIEF DESCRIPTION OF THE DRAWINGS

FIG. 1 is a perspective view of an example of a disposable absorbent pant.

FIG. 2 is a schematic plan view of an example of a precursor structure of a disposable absorbent pant shown opened and laid out flat and stretched out to its full dimensions against any contraction induced by included elastic members, with outward-facing surfaces thereof facing the viewer.

FIG. 3A is a schematic longitudinal cross-sectional, exploded view of the structure of FIG. 2 in one possible alternative configuration, taken through line 3-3 shown in FIG. 2.

FIG. 3B is a schematic longitudinal cross-sectional, exploded view of the structure of FIG. 2 in another possible alternative configuration, taken through line 3-3 shown in FIG. 2.

FIG. 3C is a schematic longitudinal cross-sectional, exploded view of the structure of FIG. 2 in another possible alternative configuration, taken through line 3-3 shown in FIG. 2.

FIG. 4 is a schematic lateral cross-sectional, exploded view of the structure of FIG. 2 in one possible configuration, taken through line 4-4 shown in FIG. 2.

FIG. 5 is a schematic plan view of another example of a precursor structure of a disposable absorbent pant shown opened and laid out flat and stretched out to its full dimensions against any contraction induced by included elastic members, with outward-facing surfaces thereof facing the viewer.

### DESCRIPTION OF EXAMPLES

### Definitions

The following definitions of the following terms apply for purposes herein:

“Absorbent article” means a disposable diaper or disposable absorbent pant.

“Cross direction” (CD)—with respect to the making of a nonwoven web material, the nonwoven material itself, a laminate thereof, or an article in which the material is a component, refers to the direction along the material substantially perpendicular to the direction of forward travel of the material through the manufacturing line in which the material and/or article is manufactured.

Throughout the present description, a material or composite of materials is considered to be “elastic” or “elastomeric” if, when a biasing force is applied to the material, the material or composite can be extended to an elongated length of at least 150% of its original relaxed length (i.e. can extend at least 50%), without rupture or breakage which substantially damages the material or composite, and when the force is removed from the material or composite, the material or composite recovers at least 40% of such elongation. In various examples, when the force is removed from an elastically extensible material, the material or composite may recover at least 60% or even at least 80% of its elongation.

“Film” means a skin-like or membrane-like layer of material formed of one or more polymers, which does not have a form consisting predominately of a web-like structure of consolidated polymer fibers and/or other fibers.

“Fine fibers” means fibers having an average diameter of 0.10 μm to 10 μm. Fine fibers may be produced by processes including, for example, meltblowing processes.

The “front waist region” of an absorbent article is the portion of the article extending longitudinally from the front waist edge to a lateral line tangent to both the left and right front leg edges, and closest to the front waist edge.

The “rear waist region” of an absorbent article is the portion of the article extending longitudinally from the rear waist edge to a lateral line tangent to both the left and right rear leg edges and closest to the rear waist edge.

The “crotch region” of an absorbent article is the portion of the article lying longitudinally between the front waist region and the rear waist region.

With respect to a pant precursor structure or diaper in an opened configuration, laid out flat and stretched out to its full dimensions against any contraction induced by included elastic members, the “longitudinal” direction is the direction perpendicular to the waist edges, and the “lateral” direction is the direction parallel to the waist edges. With respect to a wearer, the “longitudinal” direction is the direction parallel to the wearer's standing height, and the “lateral” direction is the direction perpendicular to the wearer's standing height and extending along the left-right direction relative to the wearer. References to a “length” dimension refer to a dimension measured in the longitudinal direction; references to a “width” dimension refer to a dimension measured in the lateral direction.

“Machine direction” (MD)—with respect to the making of a nonwoven web material, the nonwoven material itself, or a laminate thereof, refers to the direction along the material or laminate substantially parallel to the direction of forward travel of the material or laminate through the manufacturing line in which the material or laminate is manufactured.

“Machine direction bias,” with respect to the fibers forming a nonwoven web, means that a majority of the fibers, as situated in the web and unstretched, have lengths with machine direction vector components that are greater than their cross direction vector components.

A “nonwoven” is a manufactured sheet or web of directionally or randomly oriented fibers which are first formed into a batt and then consolidated and bonded together by friction, cohesion, adhesion or one or more patterns of bonds and bond impressions created through localized compression and/or application of pressure, heat, ultrasonic or heating energy, or a combination thereof. The term does not include fabrics which are woven, knitted, or stitch-bonded with yarns or filaments. The fibers may be of natural and/or man-made origin and may be staple and/or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes including but not limited to meltblowing, spunbonding, spunmelting, solvent spinning, electrospinning, carding, film fibrillation, melt-film fibrillation, airlaying, dry-laying, wetlaying with staple fibers, and combinations of these processes as known in the art. The basis weight of nonwoven fabrics is usually expressed in grams per square meter (gsm).

With respect to a seam joining two sections of material, “permanent” means that the sections are joined in such a manner and/or by such a mechanism that a forcible separation of the materials at the seam cannot occur without substantial damage to the article and/or the materials cannot be rejoined at the seam (without unusual measures) to substantially restore the pre-separation configuration and structural integrity of the article. Non-limiting examples of mechanisms by which a permanent seam may be formed include adhesive and thermal bonding between the sections. Conversely, a “refastenable” seam joins the sections in such a manner and/or by such a mechanism that a forcible separation of the materials at the seam can occur without substantial damage to the article, and the materials can be rejoined at the seam (without unusual measures) to substantially restore the pre-separation configuration and structural integrity of the article. A non-limiting example of mechanisms by which a refastenable seam may be formed includes inclusion of hook-and-loop fastening system component(s) to join the sections.

“z-direction” means the direction orthogonal to an x-y plane occupied or approximately defined by a pant precursor structure when laid out flat; and also means the direction orthogonal to the wearer's body surfaces (i.e., orthogonally toward or away from the wearer's body surfaces) when the pant is worn, in areas of the wearer's body covered by the pant. “z-direction,” with respect to a web, means generally orthogonal or perpendicular to the plane approximated by the web along the machine and cross direction dimensions.

All dimensions, dimensional relationships and surface areas that are referred to herein are measured with the absorbent article (or subject component thereof) opened (separated at side seams if necessary), laid out horizontally on a flat surface, and extended out to its full dimensions against any contraction induced by the presence of pre-strained elastic members.

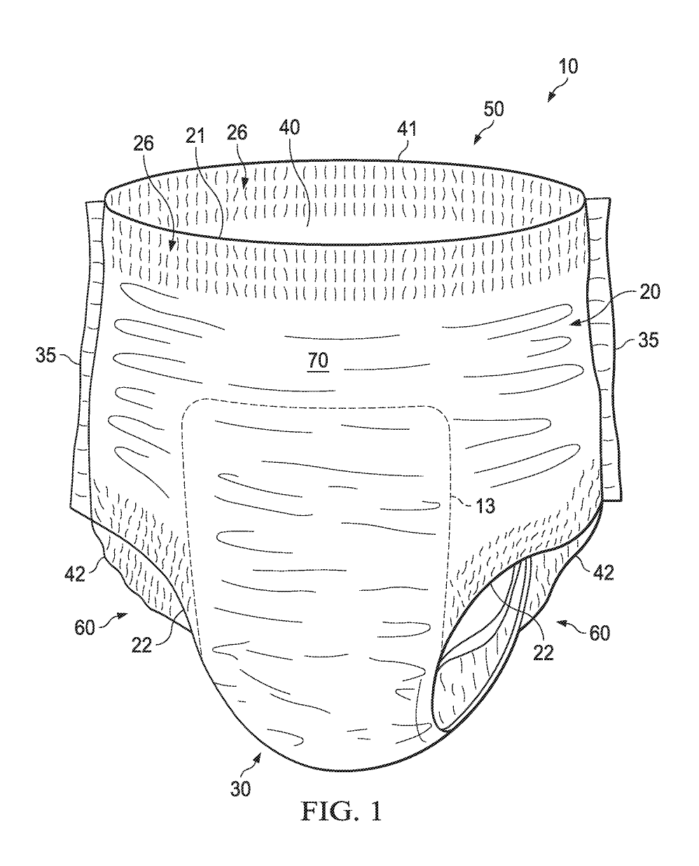

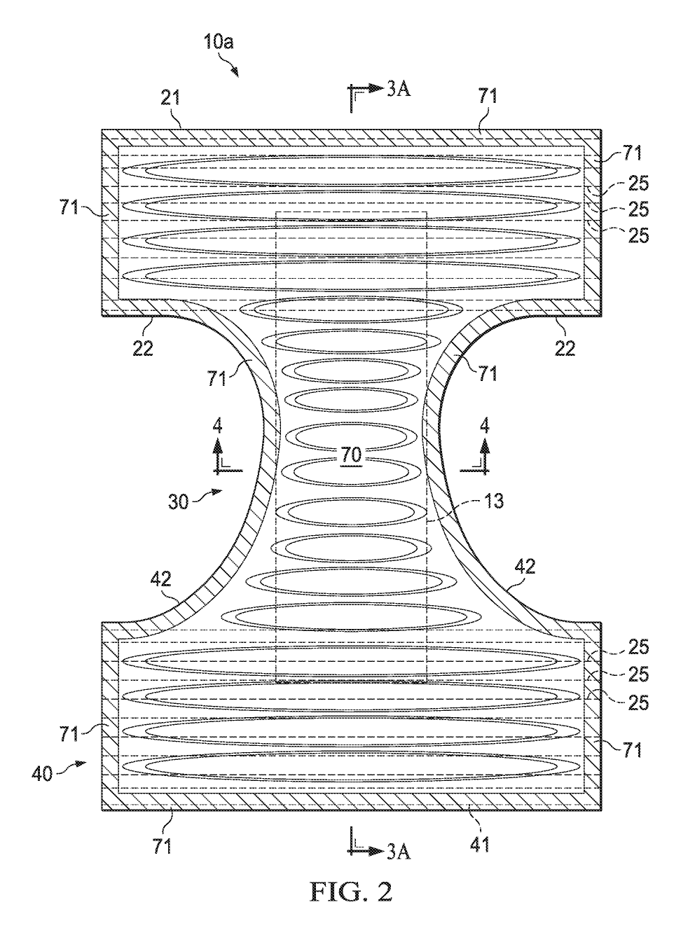

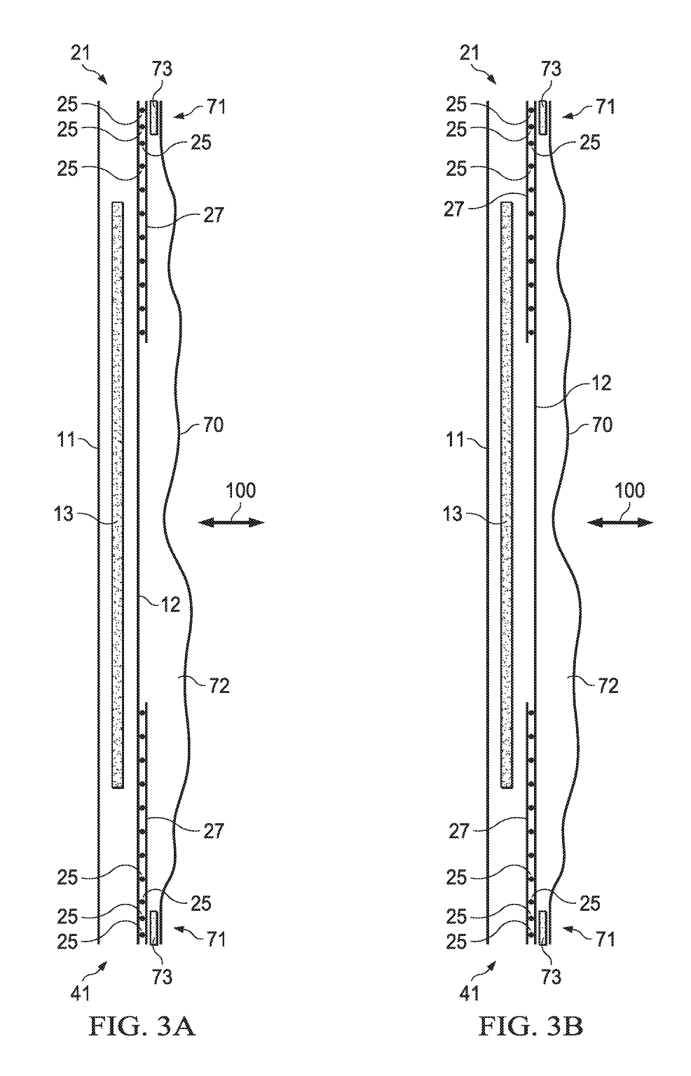

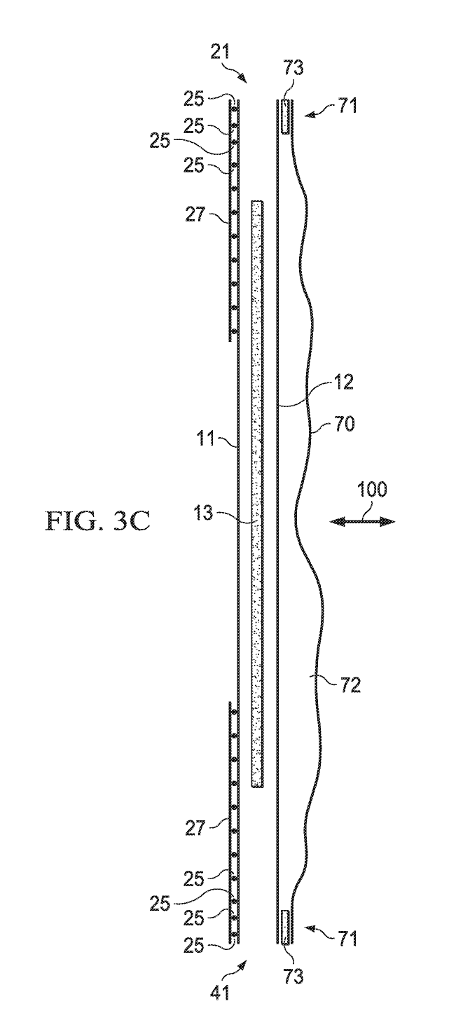

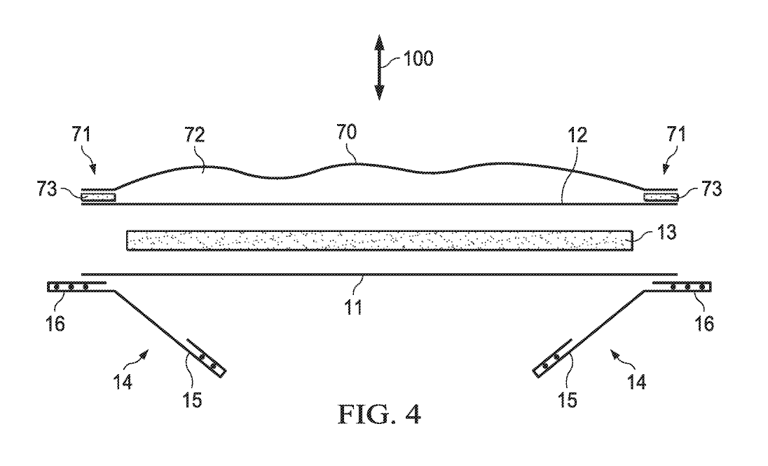

FIG. 1 is a perspective view of an example of a disposable absorbent pant 10. FIG. 2 is a schematic plan view of an example of a precursor structure of a disposable absorbent pant, with outward-facing surfaces thereof facing the viewer. FIG. 3 is a schematic longitudinal cross-sectional, exploded view of the structure of FIG. 2, taken through line 3-3 shown in FIG. 2. FIG. 4 is a schematic lateral cross-sectional, exploded view of the structure of FIG. 2, taken through line 4-4 shown in FIG. 2.

A pant 10 and its precursor structure 10a will have a front waist region 20, crotch region 30 and rear waist region 40, identified as such by the portions of the wearer's lower torso that they cover when the pant is worn. The pant structure will have lateral front waist edge 21 and lateral rear waist edge 41 defining a waist opening 50. The pant structure will have a pair of front leg edges 22 and a pair of rear leg edges 42 respectively defining a pair of leg openings 60. To form a finished pant 10, the precursor structure 10a may be folded laterally to bring respective waist edges 21, 41 toward each other, and the materials respectively forming the front and rear waist regions 20, 40 of precursor structure 10a may be joined by side seams 35 to form a pant structure. Side seams 35 may be permanent (as exemplified by side seams of current PAMPERS EASY UPS children's training pants (product of The Procter & Gamble Company, Cincinnati, Ohio), in which respective materials of the front and rear waist regions are thermally bonded together, or refastenable (as exemplified by side seams of current HUGGIES PULL-UPS children's training pants (product of Kimberly Clark Corporation, Irving, Tex.), in which respective materials of the front and rear waist regions are joined by a reusable mechanical fastening system (hook and loop system).

Referring to FIGS. 3 and 4, pant 10 may have a liquid permeable topsheet 11, a liquid impermeable backsheet 12, and an absorbent core structure 13 disposed between them. The topsheet and backsheet may be joined about their perimeters by, e.g., a pattern of thermal bonds and/or adhesive, to form an enveloping structure that contains the absorbent core structure 13. Pant 10 may have a pair of longitudinal cuff structures 14. Cuff structures may include integral or separate barrier cuffs 15 and leg cuffs 16. Barrier cuffs may be configured so as to extend away from the pant structure and toward the wearer's body surfaces through the crotch region in the z-direction 100 when the pant is worn, so as to help contain the wearer's exudates. Leg cuffs may be configured to cause the pant to fit snugly about the wearer's legs.

Non-limiting examples of suitable configurations of topsheet, backsheet, absorbent core structure and barrier cuffs are described in in U.S. patent application Ser. No. 13/764,945.

Pant 10 may include an elasticized belt configuration. An elasticized belt configuration may be formed, for example, by a plurality of longitudinally-spaced elastic strands 25 extending laterally across the front and rear waist regions. Elastic strands 25 may be disposed to the outside of the backsheet 12, and may be affixed in place at side seams 35 and/or along their lateral lengths by, e.g., strand-coating with adhesive during manufacture, or otherwise by a pattern of adhesive applied to the backsheet during manufacture of the pant. Elastic strands 25 may be incorporated into the structure in a pre-strained condition, such that, upon completion of manufacture of the pant, and relaxation of the structure, the elastic strands 25 are allowed to contract, laterally drawing the backsheet material to form ruffles or gathers of material 26 (FIG. 1). The gathers and pre-strained elastic strands impart the waist regions to with elastic stretch capabilities, facilitating donning and removal of the pant and providing for snug and secure fit during wear. A separate belt layer 27 may be included to provide a structure to sandwich the strands between the backsheet and the belt layer, helping to keep the strands in place within the structure. In alternative configurations shown in FIGS. 3A, 3B and 3C, elastic strands 26 may be disposed to the outer/garment-facing side of the backsheet 12 (FIG. 3A), to the inner/wearer-facing side of the backsheet 12 (FIG. 3B) or even to the inner/wearer-facing side of the topsheet 11 (FIG. 3C). Correspondingly, belt layer 27 may be disposed as shown in the figures. In yet another alternative, elastic strands 26 may be disposed between the backsheet 12 and topsheet 11 as suggested by FIG. 3B, and a separate belt layer may be deemed unnecessary because the elastic strands are sandwiched in place in such internal position between these layers. The elastic strands may be held in place, and the sandwiching layers held together about them, by, e.g., deposits of adhesive between the layers, thermal bonding between the layers, etc. In configurations such as shown in FIGS. 3B and 3C, the blouse layer 70 may be attached directly to the outer/garment-facing side of backsheet 12 at attachment zones 71.

As an alternative to or substitute for elastic stands 26, elasticity may be provided by one or more strips of elastic film, pre-strained in the lateral direction.

Blouse Layer

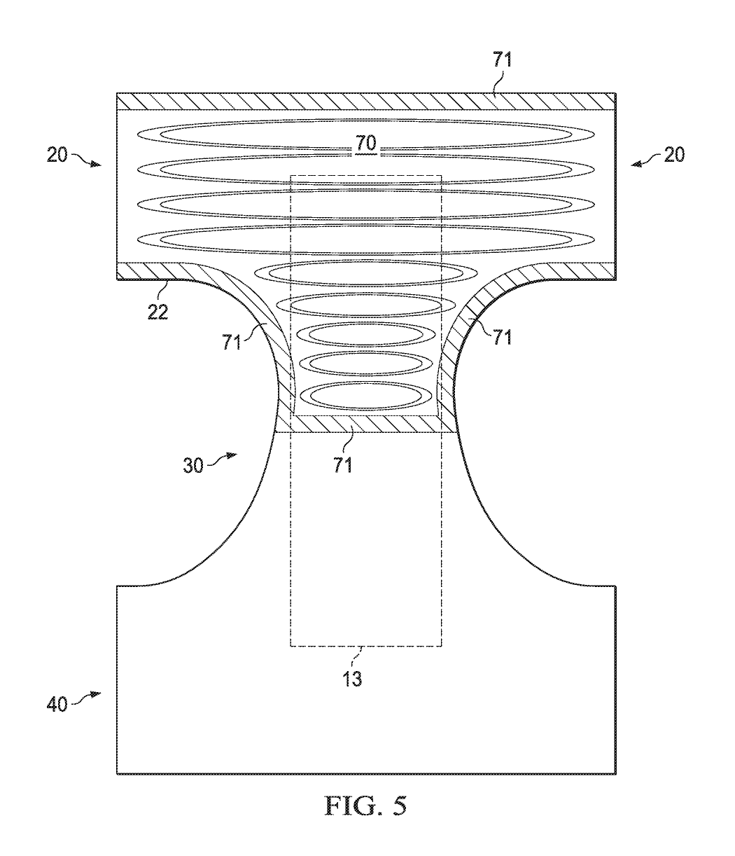

The pant 10 may be provided with an outer blouse layer 70, which overlies a portion, a substantial portion, or even the entirety of the pant structure. Outer blouse layer 70 may be formed of a fibrous nonwoven web that is highly vapor permeable. Outer blouse layer 70 may be attached about its perimeter to the pant structure at attachment zones 71 (indicated by heavy cross-hatching in FIGS. 2 and 5). An attachment zone may be disposed in the waistband region proximate the waist edge of either or both the front and rear waist regions. (Herein, the “waistband region” is the region lying within 25 mm of the waist edge.) An attachment zone may be proximate a leg opening in the leg band region. (Herein, the “leg band region” of the blouse layer is the region lying within 25 mm of the leg opening edge.) An attachment zone may be disposed partially or entirely outside the portion of the plan surface area of the underlying article structure occupied by the absorbent core structure. It may be desired in some circumstances, for maximized blousing behavior, that no attachment zones lie over the absorbent core structure, or least that no attachment zones in the front and rear waist regions lie over the absorbent core structure. Side seams 35 (see FIG. 1) may also form, or be substantially coextensive with, blouse layer attachment zones at the sides of the article. It may be preferred that attachment zones 71 substantially entirely circumscribe the blouse layer within its perimeters, so that no substantial lengths of unbonded portions of the blouse layer are present proximate its perimeter such as may create an unfinished, sloppy appearance or even the appearance of a defect. (It is noted, however, that a relatively small margin of unbonded material laterally and/or longitudinally outside an attachment zone may be desired, in some instances, for example, to impart a frilled appearance.)

Attachment between blouse layer 70 and the remaining portions of the pant, at the attachment zones, may be effected by deposits of adhesive 73 (e.g. a suitable hot melt adhesive of a type typically used to adhere components for articles of the kind), by thermal bonding, or by a combination thereof. Blouse layer 70 may be unattached to the article in regions lying longitudinally and laterally inward of the attachment zones 71 and side seams 35, thereby permitting those regions to separate and move away from the structure in the z-direction (blouse), leaving an air space 72 between blouse layer 70 and backsheet 12, and serving to impart an apparently looser and more comfortable appearance to the otherwise snug-fitting pant structure; or to impart the appearance of an over-garment. Without intending to be bound by theory, it is believed that some caregivers may prefer such an appearance.

In order to further enhance such appearance, the material forming blouse layer 70 may have a length and/or width, or a surface area, that exceed one or more of those of the underlying portions of the pant precursor structure 10a to which blouse layer 70 is attached. Herein, the dimensions and surface area of the blouse layer are expressed herein as those of the blouse layer when separated from the remaining components of the article, extended to their full dimensions and in a flat configuration. The dimensions and surface area of the underlying portions of the article are also expressed herein as those of the components when extended to their full dimensions against any contraction induced by the presence of pre-strained elastic members, in a flat configuration such as appears in FIG. 2, and will be referred to herein as the “plan” dimensions. Such greater dimensions and/or surface area provide extra material that may enhance the blousing behavior of the blouse layer 70. The blouse layer 70 may be sized and configured to have a surface area that exceeds the plan surface area by at least 5 percent, 10 percent, 15 percent or even 20 percent.

Alternatively, the blouse layer 70 may have the same dimensions as the underlying portions of the article, but the article may be assembled such that the perimeter of the blouse layer 70 and its attachment zones lie longitudinally and/or laterally inward of the perimeter of the underlying portions of the article—to similar effect.

As noted, the blouse layer 70 may be unattached to the article in regions lying longitudinally and/or laterally inward of the attachment zones 71 and side seams 35. For maximized blousing behavior, it may be desired that portions of the blouse layer 70 that are attached to the article (at attachment zones) have a combined surface area that is no more than 50 percent, 40 percent, 30 percent, 20 percent or most preferably even no more than 15 percent of the total surface area of the blouse layer (when the blouse layer is separated from the remainder of the pant structure and fully extended).

A pattern of apertures in the blouse layer may further enhance the visual appeal of the article. When a pattern of apertures is included, as the material of the blouse layer moves and shifts with the wearer's movements, the apertures will move and shift relatively the underlying backsheet, which may impart an appearance of depth, movement and complexity. Suitable examples of apertures in nonwoven webs, and aperture forming methods, are disclosed and/or identified by reference in co-pending applications publications nos. US 2015/0088088 and US 2015/0083310.

The visual effect may be further enhanced if the material(s) forming the blouse layer 70 and the material(s) forming the backsheet 12 or other layer immediately underlying the blouse layer 70 have visually contrasting colors. In one example, the material(s) forming the blouse layer may impart a white or nearly white color to the blouse layer. Material(s) forming the backsheet, or printing thereon, or tinting or pigmenting materials incorporated thereinto, may impart the backsheet with one or more colors that perceivably contrast white. The contrast between a non-white backsheet 12 and an overlying white, apertured blouse layer 70, can impart an interesting, lively, deep, complex, even shimmering appearance to the outer surfaces of the blouse layer when article when worn.

Alternatively or in addition, the material(s) forming the backsheet 12 may impart a substantially white color, or a singular color to the backsheet or other underlying layer, while the material(s) forming the blouse layer may be printed in any decorative images or patterns in color(s) that visually contrast with the color of underlying backsheet materials as seen through the apertures. It may be preferred that any such printing be disposed on the wearer-facing, inward-facing surface of the blouse layer, to help protect the printing from abrasion.

The material of the blouse layer and underlying layer one or both layers may be tinted, pigmented or printed in one or more colors or shades (including white) such that the colors or shades of the respective layers visually contrast. The contrasting color or shade of the underlying layer can then be seen through apertures of the blouse layer for interesting visual effect. Herein, a “visual contrast” between colors or shades of two respective layers of material means that the value of delta E* determined through the Visual Contrast method below is equal to or greater than 2.0. For enhanced visual contrast, it may be preferred that the value of delta E* be equal to or greater than 3.5.

Nonwoven web materials of the type typically used to form such belts are generally highly breathable. (Breathability, typically reflected in measurable vapor permeability of the material, is desired to avoid overhydration of the wearer's skin beneath the article.) Accordingly, it not necessary or desirable to provide apertures merely for the purpose of increasing breathability. Because the materials are already highly breathable aperturing may have little effect in this regard. However, it is believed that the visible presence of apertures in the material may in some circumstances give consumers the impression of high breathability, or reinforce or increase such impression—which may provide a marketing advantage for the manufacturer.

In addition to imparting appearance features, an outer layer that is configured to blouse may enhance breathability of the article. As the wearer moves and shifts, the blouse layer 70 will also move and shift, causing air to move in and out of the air space 72 between the blouse layer, thereby providing ventilation of vapors collecting outside the backsheet 12. The effect may be enhanced if the blouse layer 70 is apertured, increasing its vapor permeability.

As reflected in FIG. 5, a blouse layer 70 may be included over only a portion of the pant structure. In the example shown in FIG. 5, only the front waist region and forward portion of the crotch region include a blouse layer 70. In other alternatives (not shown), a blouse layer may be included in only the front waist region, only the rear waist region, or only the front and rear waist regions but not the crotch region.

Blouse Layer Nonwoven

Blouse layer 70 may be formed of nonwoven web. Suitable nonwoven web materials that may be useful in the present invention include, but are not limited to spunbond, spunlaid, meltblown, spunmelt, solvent-spun, electrospun, carded, film fibrillated, melt-film fibrillated, air-laid, dry-laid, wet-laid staple fibers, and other and other nonwoven web materials formed in part or in whole of polymer fibers, typically used as components of disposable diapers and disposable absorbent pants. The nonwoven web may be formed predominately of polymeric fibers. In some examples, suitable non-woven fiber materials may include, but are not limited to polymeric materials such as polyolefins, polyesters, polyamide, or specifically, polypropylene (PP), polyethylene (PE), poly-lactic acid (PLA), polyethylene terephthalate (PET) and/or blends thereof. In some examples, the fibers may be formed of PP/PE blends such as described in U.S. Pat. No. 5,266,392. Nonwoven fibers may be formed of, or may include as additives or modifiers, components such as aliphatic polyesters, thermoplastic polysaccharides, or other biopolymers. Further useful nonwovens, fiber compositions, formations of fibers and nonwovens and related methods are described in U.S. Pat. Nos. 6,645,569; 6,863,933; and 7,112,621; and in co-pending U.S. patent application Ser. Nos. 10/338,603; 10/338,610; and Ser. No. 13/005,237.

The individual fibers may be monocomponent or multicomponent. The multicomponent fibers may be bicomponent, such as in a core-and-sheath or side-by-side arrangement. Often, the individual components comprise polyolefins such as polypropylene or polyethylene, or their copolymers, polyesters, thermoplastic polysaccharides or other biopolymers.

According to one example, the nonwoven may comprise a material that provides good recovery when external pressure is applied and removed. Further, according to one example, the nonwoven may comprise a blend of different fibers selected, for example from the types of polymeric fibers described above. In some embodiments, at least a portion of the fibers may exhibit a spiral curl which has a helical shape. According to one example, the fibers may include bicomponent fibers, which are individual fibers each comprising different materials, usually a first and a second polymeric material. It is believed that the use of side-by-side bi-component fibers is beneficial for imparting a spiral curl to the fibers.

To enhance loft and promote perceptions of softness of the blouse layer, the nonwoven may be treated by hydrojet impingement, which may also be known as hydroenhancement, hydroentanglement or hydroengorgement. Such nonwovens and processes are described in, for example, U.S. Pat. Nos. 6,632,385 and 6,803,103, and U.S. Pat. App. Pub. No. 2006/0057921, the disclosures of which are incorporated herein by reference.

Other examples of nonwoven web that may be useful for the blouse layer may be an SMS web (spunbond-meltblown-spunbond web) made by Avgol Nonwovens LTD, Tel Aviv, Israel, under the designation XL-S70-26; a softband SSS (spunbond-spunbond-spunbond) web made by Pegas Nonwovens AS in Znojmo, Czech Republic, under the designation 18 XX 01 00 01 00 (where XX=the variable basis weight); an SSS web made by Gulsan Sentetik Dok San VE TIC AS, in Gaziantep, Turkey, under the designation SBXXF0YYY (where XX=the variable basis weight, and YYY=the variable cross direction width); an HESB (hydroenhanced spunbond) web made by First Quality Nonwovens Inc., in Hazelton, Pa., under the designation SEH2503XXX (where XXX=the variable cross direction width); and a bicomponent SS web.

A nonwoven web useful as a component to form a blouse layer may be pre-bonded, prior to downstream processing such as aperturing as described below. A batt of fibers may be calendered and pre-bonded in a pattern, to consolidate the batt/fibers and create a pattern of bonds that adds tensile strength and dimensional stability, converting the batt of fibers to a coherent and useable nonwoven web material. The web may be imparted with a pattern of pre-bonding as described in, for example, U.S. Pat. No. 5,916,661 (pre-bonding in a pattern of “point calendered bonds 200 to form a coherent web structure”) and co-pending U.S. application Ser. No. 13/893,405 (pattern of “primary fiber bonds”). The pre-bonding may consist of a pattern of thermal bonds, mechanical bonds or adhesive bonds, although in some circumstances thermal bonding may be preferred.

Apertured topsheets have been included in absorbent articles of the type described herein. Creating apertures in nonwoven material used to form a topsheet enhances its ability to allow aqueous liquid exudates to pass therethrough. In some circumstances this may be desired because materials of which topsheets are often formed may include polymers (such as polyolefins) that are normally hydrophobic, and pores or passageways ordinarily present between the nonwoven fibers may be insufficiently large to allow aqueous liquids to pass therethrough at a desired rate because the material tends to repel aqueous liquid.

An example of a process for creating apertures in a pre-bonded nonwoven web to be used to form a blouse layer is described in U.S. Pat. Nos. 5,916,661 and 5,629,097. This process involves rolling the pre-bonded nonwoven web through the nip between a pair of rollers, one of which bears a pattern of raised bonding protrusions, and supplying heating energy to heat the fibers beneath the protrusions in the nip. When appropriately controlled pressure and heating energy are provided at the nip, a pattern of suitable bonds or “weakened, melt-stabilized locations” having rod shapes or other shapes results. At the bond sites, the polymer fibers of the web are melted, compressed and thereby fused, such that the fused polymer material at the bond sites is relatively thin (in the z-direction) and frangible. Upon subsequent cross direction incremental stretching of the bonded nonwoven web as described in the above-cited patents, the material at the bond sites or “melt-stabilized locations” breaks and apertures open in a direction transverse to the long dimension of the rod shapes. For example, as described in U.S. Pat. App. Pub. No. 2015/0083310, a nonwoven web may be thermal/calender bonded with a bonding pattern of rod shapes having their long dimension oriented in the machine direction. Following such bonding, the web may be subjected to an incremental stretching process to stretch the web in the cross direction. When the bonding process has been appropriately controlled to create relatively thin, frangible bond sites, this causes the rod-shaped bonds to break open, creating apertures through the web. Advantageously, fibers of the nonwoven web along the edges of the apertures are fused as a result of the bonding process. In comparison to a process in which apertures are simply punched or cut through the web without application of heating energy, the bonding/stretching process described in the above-cited reference does not cut the fibers, which can result in loose fibers and fraying about the edges of the punched or cut apertures. Rather, the bonding/stretching process described tends not to create loose fibers, and provides more neatly defined edges about the apertures. Following incremental stretching, the web may be allowed to relax, which may cause the apertures to close to some extent, but they will still be present.

In another example, the web may be bonded by compression bonding without the application of externally-produced or additional heating energy. Examples of suitable compression bonding systems utilizing rollers are described in, for example, U.S. Pat. Nos. 4,854,984 and 4,919,738. In these types of mechanisms, a first roller and second roller are arranged with their axes in parallel and urged together to form a nip. The first roller may have on its surface one or more bonding protrusions arranged in a pattern. The first roller and second roller may be urged together by one or more actuators such as bellows-type pneumatic actuators acting directly or indirectly on one or both of their axles, to provide and regulate compression, beneath the protrusions at the nip, of the web material as it passes therethrough, in the manner described in the aforementioned patents. A compression bonding mechanism such as, but not limited to, the mechanism described in the aforementioned patents, provides bonding of a nonwoven web material through rapid compression of superimposed fibers beneath the bonding protrusions, along the roller nip line. Without intending to be bound by theory, it is believed that rapid compression beneath the protrusions causes the respective materials to be rapidly deformed and partially expressed together from beneath the protrusions, to form structures of deformed, compressed and entangled fiber material beneath and/or around the protrusions. Welds or weld-like structures at or about the protrusions result. In some circumstances compression bonding provides advantages, including relative simplicity and cost effectiveness. It may reduce or eliminate the need for more complex bonding systems that require a system to supply externally produced or additional heating energy. Without intending to be bound by theory, it is believed that these advantages are substantially independent of variations in line speeds in at least some circumstances, including line speeds within currently known economically and technically feasible ranges for manufacture of disposable diapers and training pants. Following such creation of compression bonds, the web may be incrementally stretched to create apertures at the bond sites, in the manner taught by U.S. Pat. No. 5,916,661.

As noted, as suggested in U.S. Pat. No. 5,916,661, prior to aperturing, the nonwoven web may be pre-bonded with a relatively dense pattern of thermal/calender bonds. Following that, a pattern of apertures may simply be punched or cut through the web. A relatively dense pattern of bonding can serve to minimize loose cut fibers and fraying, and help maintain defined edges of apertures formed by cutting or punching.

It will be appreciated that the apertures created need not necessarily be rod-shaped. Other examples of shapes and patterns are described in co-pending application Pub. No. US 2014/0336605. The apertures may be rod-shaped, arc-shaped, other curved finite paths, circular, oval, elliptical or polygon, and any combinations thereof. It may be desired in some circumstances as suggested in the figures, however, that the longest dimension of a majority of the individual apertures be oriented along the machine direction of the nonwoven web—particularly when the web or components of it are formed by processes that produce a machine direction bias in the fibers such as spunbonding or spunlaying processes. (For purposes herein, “oriented along the machine direction” means that the machine direction vector component of the longest dimension of an aperture is greater than the cross direction vector component.) Because of such fiber orientation, this reduces chances that sections of fibers between adjacent apertures along the machine direction will fray or tear away. At the same time, however, while it may be desired in some circumstances that the longest dimension of a majority of the apertures be oriented along the machine direction, it may also be desired that the longest dimension is not parallel with the machine direction. In one example, in which the apertures are elliptical or oval-shaped, it may be desired that their longest dimensions are oriented along angle(s) a between greater than 0 and less than 45 degrees of the machine direction. It will be appreciated that this may add to visual and actual texturing effects, by causing the material along the edges of the apertures to move in a more complex manner in the machine, cross and z-directions as the belt is stretched and moved as during wear. It will also be appreciated that the apertures may be arranged in varying patterns, such as but not limited evenly-spaced and aligned rows and columns, offset rows and columns, diagonal patterns, shaped patterns, etc.

Additionally, the pattern of the apertures may be substantially similar or identical to the pattern of the pre-bonds (if present), in one or more of machine-direction spacing, cross-direction spacing, aperture shape and aperture size. For example, a pattern of pre-bonds may have substantially similar machine and cross direction spacing as the pattern of apertures. Using respective patterns of pre-bonds and apertures that are substantially similar in one or more respects noted can help give the material a more uniform, orderly and/or coherent appearance, and may also help enhance tensile strength as compared with a web in which respective patterns of pre-bonds and apertures do not have such similarities.

Topsheet

The topsheet 11 may be joined to the absorbent core structure 13 and/or the backsheet 12. It should be recognized that other structures, elements, or substrates may be positioned between the core structure 13 and the topsheet 11 and/or backsheet 12. While the topsheet 11, the backsheet 12, and the absorbent core structure 12 may be assembled in a variety of configurations, examples are described generally in U.S. Pat. Nos. 3,860,003; 5,151,092; 5,221,274; 5,554,145; 5,569,234; 5,580,411; and 6,004,306.

The topsheet 11 is generally a portion of the article that may be positioned at least in partial contact or close proximity to a wearer. Suitable topsheets 11 may be manufactured from a wide range of materials, such as porous foams; reticulated foams; apertured plastic films; or woven or nonwoven webs of natural fibers (e.g., wood or cotton fibers), synthetic fibers (e.g., polyester, polyolefin e.g. polyethylene or polypropylene fibers), or a combination of natural and synthetic fibers. The topsheet 11 is generally supple, soft feeling, and non-irritating to a wearer's skin. Generally, at least a portion of the topsheet 11 is liquid pervious, permitting liquid to readily penetrate through its thickness. One topsheet material useful herein is available from BBA Fiberweb, Brentwood, Tenn. as supplier code 055SLPV09U.

Any portion of the topsheet 11 may be coated with a lotion or skin care composition. Examples of suitable lotions include those described in U.S. Pat. Nos. 5,607,760; 5,609,587; 5,635,191; and 5,643,588. The topsheet 11 may be fully or partially elasticized or may be foreshortened so as to provide a void space between the topsheet 11 and the core structure 13. Exemplary structures including elasticized or foreshortened topsheets are described in more detail in U.S. Pat. Nos. 4,892,536; 4,990,147; 5,037,416; and 5,269,775.

Backsheet

The backsheet 12 is generally positioned to the garment-facing/outward-facing side of the absorbent core structure. Backsheet 12 may be designed to prevent the exudates absorbed by and contained within the pant from soiling articles that may contact the pant, such as bed sheets or outer clothing. In some examples, the backsheet 12 is effectively liquid-impermeable. Suitable backsheet 12 component materials include films such as those manufactured by Tredegar Industries Inc. of Terre Haute, Ind. and sold under the trade names X15306, X10962, and X10964.

The ventilation/breathability effect of including a blouse layer 70 may be further enhanced if backsheet 12 is formed of vapor permeable/breathable web material. In one example, backsheet 12 may be formed of a vapor permeable film, by way of non-limiting example, such as disclosed in U.S. Pat. Nos. 7,307,031; 6,677,258; and 6,429,352. Other suitable backsheet component materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746 and U.S. Pat. No. 5,865,823. Other breathable backsheets including nonwoven webs and apertured formed films are described in U.S. Pat. No. 5,571,096. An exemplary, suitable backsheet is disclosed in U.S. Pat. No. 6,107,537. Other suitable materials and/or manufacturing techniques may be used to provide a suitable backsheet 12 including, but not limited to, surface treatments, particular film selections and processing, particular fiber selections and processing, etc.

Backsheet 12 may also consist of more than one layer. The backsheet 12 may comprise an outer cover and an inner liquid barrier layer. The outer cover may be made of a nonwoven web material. The liquid barrier layer may be made of a substantially liquid-impermeable film. The backsheet may be a laminate of the outer cover and the liquid barrier layer, wherein the layers are held to together, e.g., by a pattern of applied adhesive, e.g., a hot melt adhesive of the type commonly used in the absorbent article manufacturing industry. The surface area of the liquid barrier layer may be smaller than that of the outer cover. In another example the liquid barrier layer may be made of a substantially liquid-impermeable nonwoven, for example, a nonwoven formed at least in part of microfibers or nanofibers having a combination of hydrophobicity and numeric density per unit surface area sufficient to make the nonwoven effectively liquid impermeable under normal use conditions. The outer cover and an liquid barrier layer may be joined together by adhesive or any other suitable material or method. A particularly suitable outer cover is available from Corovin GmbH, Peine, Germany as supplier code A18AH0, and a particularly suitable inner layer is available from RKW Gronau GmbH, Gronau, Germany as supplier code PGBR4WPR. While a variety of backsheet configurations are contemplated herein, various other changes and modifications can be made without departing from the spirit and scope of the invention.

In another alternative, backsheet 12 may be highly vapor permeable yet liquid impermeable because it comprises or is formed of a layer of densely spaced polymeric fine fibers such as disclosed in, by way of non-limiting example, U.S. App. Pub. No. US 2011/0196327.

Absorbent Core Structure

The absorbent core structure 13 includes the entirety of the structure and components thereof disposed between the topsheet and the backsheet, and may comprise a wide variety of liquid-absorbent materials commonly used in disposable diapers and other absorbent articles. Examples of suitable absorbent materials include comminuted wood pulp, which is generally referred to as air felt creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

In one example, at least a portion of the absorbent core structure 13 is substantially cellulose free and contains less than 10% by weight cellulosic fibers, less than 5% cellulosic fibers, less than 1% cellulosic fibers, no more than an immaterial quantity of cellulosic fibers or no cellulosic fibers. It should be understood that an immaterial quantity of cellulosic material does not materially affect at least one of the thinness, flexibility, and absorbency of the portion of the absorbent core structure that is substantially cellulose free. Among other benefits, it is believed that when at least a portion of the absorbent core structure is substantially cellulose free, this portion of the absorbent core structure is significantly thinner and more flexible than a similar absorbent core structure that includes more than 10% by weight of cellulosic fibers. The amount of absorbent material, such as absorbent particulate polymer material present in the absorbent core structure may vary, but in certain embodiments, is present in the absorbent core structure in an amount greater than about 80% by weight of the absorbent core structure, or greater than about 85% by weight of the absorbent core structure, or greater than about 90% by weight of the absorbent core structure, or greater than about 95% by weight of the core. Exemplary absorbent structures for use as the absorbent core structure 13 are described in U.S. Pat. Nos. 4,610,678; 4,673,402; 4,834,735; 4,888,231; 5,137,537; 5,147,345; 5,342,338; 5,260,345; 5,387,207; 5,397,316; and 5,625,222.

The absorbent core structure 13 and components thereof also may be constructed to provide a system of substantially longitudinally-oriented channels as disclosed in, for example, U.S. application Ser. Nos. 13/491,642; 13/491,644; 13/675,212; 13/709,169; 13/709,244; 13/709,254; and Ser. No. 14/077,355. As noted in the cited applications, a system of one or more substantially longitudinally-oriented channels in the absorbent core structure provides for efficient liquid distribution across the absorbent structure, and also a relatively thinner and more flexible core structure, contributing to an overall sleek, low-bulk, underwear-like look and feel to the pant structure. The channels are grooves or valleys defined through the absorbent material of the core. They may perform at least two functions, including providing passageways along which liquid may rapidly flow to reach and contact surface area of more absorbent material along the length of the absorbent core structure, and providing hinge- or joint-like structures in the absorbent core structure along which the absorbent core structure may more easily flex, providing comfort and bulk-reducing effects.

Cuff Structures

Referring to FIG. 4, the article may generally have any structure that is suitable for disposable absorbent articles such as diapers and training pants, including any of the absorbent core structure and leg cuff/gasketing structures described and depicted in U.S. application Ser. No. 13/457,521, and including barrier 15 and leg 16 cuff portions of cuff structures 14.

Elastic Members

Elastic strands 25 may be formed of an elastomeric material, such as an elastane (for example, LYCRA HYFIT fiber, a product of Invista, Wichita, Kans.). Layers of the pant may be joined together about elastic strands 25 by adhesive deposited between the layers, by thermal bonds, by compression bonds, or by a combination thereof. In other examples, the one or more elastic members may be strips or a section of film formed of elastomeric material.

The elastomeric members can also be formed from various other materials, such as but not limited to, rubbers, styrene ethylbutylene styrene, styrene ethylene propylene styrene, styrene ethylene ethylene propylene styrene, styrene butadiene styrene, styrene isoprene styrene, polyolefin elastomers, elastomeric polyurethanes, and other elastomeric materials known in the art, and combinations thereof. In some embodiments, the elastic members can be extruded strand elastics with any number of strands (or filaments). The elastomeric members can have a decitex ranging from 50 to 2000, or any integer value for any decitex value in this range, or any range formed by any of these integer values. The elastomeric members may be in a form of film. Examples of films have been described extensively in prior patent applications (see, for example, U.S. Pat. App. Pub. No. 2010/0040826). The film may be created with a variety of resins combined in at least one of several sublayers, the latter providing different benefits to the film.

During manufacture of pant, the elastic strands 25 or other elastic member(s) may be strained in a lateral direction (relative the pant) by a desired amount as they are being incorporated into the structure. Upon subsequent relaxation of the structure, the elastic member(s) such as elastic strands 25 will contract toward their unstrained lengths. This causes the sandwiching layers to gather and form ruffles or gathers 26 having ridges and valleys extending generally transverse to the direction of pre-strain. This also enhances or increases the blousing behavior of the blouse layer 70. In one possible combination, the majority of the elasticized region of the pant structure underlying the blousing layer in the front and rear waist regions is unattached to the blousing layer. Expressed differently, the attachment zones in the front and rear waist regions may overlie only a minority fraction of the surface area of the elasticized regions of the pant structure in the front and rear waist regions. (For purposes herein, an “elasticized region” is a region defined by the outline of an included continuous section of elastic film, or a region delineated by the longitudinally outermost edges of two elastic members of a group of two or more elastic members longitudinally spaced less than 15 mm apart.) In some circumstances this may be preferred because it reduces the appearance of wrinkles in the blouse layer 70 that may result from being joined to the structure at areas overlying ruffles or gathers resulting from contraction of pre-strained elastic members.

Visual Contrast Test Method

The color difference measurement is based on the CIE L*a*b* color system (CIELAB). A flat bed scanner capable of scanning a minimum of 24 bit color at 1200 dpi and has manual control of color management (a suitable scanner is an Epson Perfection V750 Pro from Epson America Inc., Long Beach Calif.) is used to acquire images. The scanner is calibrated against a color reflection target compliant to ANSI method IT8.7/2-1993 using color management software (a suitable package is MonacoEZColor available from X-Rite Grand Rapids, Mich.) to construct a scanner profile. The resulting calibrated scanner profile is opened within an imaging program that supports sampling in CIE L*a*b* (a suitable program is Photoshop S4 available from Adobe Systems Inc., San Jose, Calif.) to measure bonded and unbonded areas.

Turn on the scanner for 30 minutes prior to calibration. Place the IT8 target face down onto the scanner glass and close the scanner lid. Open the MonacoEZColor software and select acquire image using the Twain software included with the scanner. Within the Twain software deselect the unsharp mask setting and any automatic color correction or color management options that may be included in the software. If the automatic color management cannot be disabled, the scanner is not appropriate for this application. Acquire a preview scan at 200 dpi and 24 bit color. Insure that the scanned image is straight and first outer surface facing side-up. Crop the image to the edge of the target, excluding all white space around the target, and acquire the final image. The MonacoEZColor software uses this image to compare with included reference files to create and export a calibrated color profile compatible with Photoshop. After the profile is created the scan resolution (dpi) can be changed, but all other settings must be kept constant while imaging samples.

Provide respective samples of each layer 75 mm by 75 mm square. Precondition the samples at about 23° C.±2 C.° and about 50%±2% relative humidity for 2 hours prior to testing.

Open the scanner lid and place the first sample onto the scanner glass with the first outer surface facing the glass. Cover the sample with the white background (in this test method white is defined as having L*>94, −2<a*<2, and −2<b*<2) and close the lid. Acquire and import a scan of the first sample into Photoshop at 600 dpi and 24 bit color. Assign the calibrated scanner profile to the image and change the mode to Lab Color (“Lab Color” in Photoshop corresponds to the CIE L* a* b* standard). Select the “eyedropper” color selection tool. Set the sampling size of the tool to include as many pixels as possible within an area of the sample 2 mm by 2 mm square, which does not include an aperture. Using the eyedropper tool measure and record L*a*b* values in 10 different 2 mm by 2 mm square areas (not having apertures) in the sample image. Average the 10 individual L*a*b* values and record as L₁, a₁, and b₁ respectively.

Repeat the steps in the paragraph above for the second sample, and record the averaged values as L₂, a₂ and b₂. Calculate and report the color difference (delta E*) between the bonded and unbonded areas using the following equation:

delta E*=\sqrt{(L₂*−L₁*)²+(a₂*−a₁*)²+(b₂*−b₁*)²}

and report to the nearest 0.01 units. A total of three substantially identical samples of each layer are measured for each sample set. Average the three delta E* values and report to the nearest 0.1 unit.

All patents and patent applications (including any patents which issue thereon) referred to herein are hereby incorporated by reference to the extent that it is consistent herewith.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is, therefore, intended that the scope of the invention is limited only by the appended claims and equivalents thereof.

What is claimed is:

1. An absorbent article comprising a liquid permeable topsheet, a liquid impermeable backsheet and an absorbent core structure disposed between the topsheet and backsheet, and an outer blouse layer formed of a first nonwoven web, the blouse layer having a blouse layer surface area, the blouse layer being attached to the article at attachment zones having an attachment surface area, the attachment surface area comprising less than 50 percent of the blouse layer surface area.

2. The article of claim 1 further comprising a front waist region, a rear waist region and a crotch region bridging the front waist region and the rear waist region, wherein the blouse layer overlies one or more of the front waist region, rear waist region and crotch region.

3. The article of claim 2 comprising an elastic laminate in one or both of the front and rear waist regions, the elastic laminate comprising one or more elastic members adhered to a substrate web material.

4. The article of claim 3 wherein the substrate material is any of the topsheet, the backsheet or a belt layer.

5. The article of claim 3 wherein the one or more elastic members comprise a plurality of laterally extending elastic strands.

6. The article of claim 3 wherein the one or more elastic members comprise an elastic film.

7. The article of claim 3 wherein the one or more elastic members have been incorporated into the elastic laminate in laterally pre-strained condition.

8. The article of claim 3 wherein the elastic laminate is disposed to the garment or outward-facing side of the absorbent core structure.

9. The article of claim 2 wherein the front waist region and the rear waist region have waist opening edges and waistband regions proximate the waist opening edges, and the front waist region, rear waist region and crotch region have leg opening edges and right and left leg band regions proximate the leg opening edges, and the attachment zones are at least partially present at one or more of the waistband regions and leg band regions.

10. The article of claim 9 having left and right side seams joining the front and rear waist regions to form a waist opening and left and right leg openings, and the side seams form a portion of the attachment zones or a portion of the attachment zones are co-located with the side-seams, and the blouse layer is unattached to the article except at the attachment zones.

11. The article of claim 1 wherein the blouse layer is formed of a single continuous section of nonwoven web.

12. The article of claim 1 having a plan surface area, and wherein the blouse layer overlies at least 50 percent of the plan surface area.

13. The article of claim 1 wherein the liquid impermeable backsheet comprises a polymeric film.

14. The article of claim 1 wherein the liquid impermeable backsheet comprises a second nonwoven web.

15. The article of claim 14 wherein the second nonwoven web comprises a layer formed of fine fibers.

16. The article of claim 1 wherein the blouse layer has a surface area and underlying portions of the article have a plan surface area, and the blouse layer surface area is greater than the plan surface area.

17. The article of claim 16 wherein the blouse layer surface area is at least 10 percent greater than the plan surface area.

18. The article of claim 1 wherein the attachment zones substantially entirely contain an inner portion of the blouse layer surface area.

19. The article of claim 1 wherein the liquid impermeable backsheet is vapor permeable.

20. The article of claim 1 wherein no attachment zones in the front and rear waist regions overlie the absorbent core structure.

21. The article of claim 20 wherein no attachment zones overlie the absorbent core structure.
